(19) United States
(12) Patent Application Publication
(10) Pub. No.: US 2003/0069532 A1
(43) Pub. Date: Apr. 10, 2003

(54) Transmyocardial implant with reinforcing wrap

(76) Inventors: Dave Mowry, Waconia, MN (US); Robert Kohler, Lake Elmo, MN (US); Michael O'Connor, Lino Lakes, MN (US)

Correspondence Address: Julie R. Daulton, Merchant & Gould P.C., P.O. Box 2903, Minneapolis, MN, 55402-0903, US

(21) Appl. No.: 09/972,779

(22) Filed: Oct. 5, 2001

(51) Int. Cl. A61M 5/00; A61F 2/06

(52) U.S. Cl. 604/8; 623/1.1

(57) ABSTRACT

A transmyocardial implant for establishing a blood flow path through a myocardium between a heart chamber and a lumen of a coronary vasculature residing at an exterior of the myocardium includes a hollow, rigid conduit having a wall defining an outer surface and an interior; an open first end; an open second end; a first portion defining the first end; a second portion defining the second end; and a reinforcing wrap. The first portion is dimensioned to be received within the lumen and the second portion is dimensioned to extend from the vasculature to the myocardium into the chamber. The conduit defines a blood flow pathway within the interior between the first and second ends. The second portion is formed of a material sufficiently rigid to resist deformation and closure of the pathway in response to contraction of the myocardium. Preferably, the reinforcing wrap extends along at least a section of the outer surface of the wall. In one embodiment, the reinforcing wrap is a spiral winding. In another embodiment, the reinforcing wrap is a woven braid.

### TECHNICAL FIELD

This disclosure relates to an implant for directing blood flow directly between a chamber of the heart and a coronary vasculature. This disclosure also relates to methods of use of an implant.

### BACKGROUND

U.S. Pat. No. 5,944,019, issued Aug. 31, 1999, teaches an implant for defining a blood flow conduit directly from a chamber of the heart to a lumen of a coronary vessel. An embodiment disclosed in this patent teaches an L-shaped implant in the form of a rigid conduit having one leg sized to be received within a lumen of a coronary artery and a second leg sized to pass through the myocardium and extend into the left ventricle of the heart. As disclosed in the '019 patent, the conduit is rigid and remains open for blood flow to pass through the conduit during both systole and diastole. The conduit penetrates into the left ventricle in order to prevent tissue growth and occlusions over an opening of the conduit. U.S. Pat. No. 5,944,019 is incorporated by reference herein.

U.S. Pat. No. 5,984,956, issued Nov. 16, 1999, discloses an implant with an enhanced fixation structure. The enhanced fixation structure includes a fabric surrounding at least a portion of the conduit to facilitate tissue growth on the exterior of the implant. U.S. Pat. No. 5,984,956 is incorporated herein by reference. U.S. Pat. No. 6,029,672 issued Feb. 29, 2000 teaches procedures and tools for placing a conduit. U.S. Pat. No. 6,029,672 is incorporated herein by reference.

Improvements in implants continue to be desirable.

### SUMMARY

In one aspect, a transmyocardial implant for establishing a blood flow path through a myocardium between a heart chamber and a lumen of a coronary vasculature residing at an exterior of the myocardium is provided. The implant includes a hollow, rigid conduit having a wall defining an outer surface and an interior, an open first end, an open second end, a first portion defining the first end, a second portion defining the second end, and a reinforcing wrap. The first portion is dimensioned to be received within the lumen and the second portion is dimensioned to extend from the vasculature to the myocardium into the chamber. The conduit defines a blood flow pathway within the interior between the first and second ends. The second portion is formed of a material sufficiently rigid to resist deformation and closure of the pathway in response to contraction of the myocardium. Preferably, the reinforcing wrap extends along at least a section of the outer surface of the wall.

In one embodiment, the reinforcing wrap includes a band comprising a spiral winding around at least a section of the wall of the conduit. In another embodiment, the reinforcing wrap includes a woven braid covering at least a section of the second portion.

In another aspect, a method for making a transmyocardial implant includes providing a hollow, rigid conduit having a wall defining an outer surface and an interior, an open first end, an open second end, a first portion defining the first end, and a second portion defining the second end. The method includes providing a reinforcing wrap to extend along at least a section of the outer surface of the wall.

In another aspect, a method for performing a coronary vessel bypass procedure for supplementing a flow of blood to a coronary vessel is provided. The method includes forming a blood flow path from a heart chamber directly to the coronary vessel at a site in the vessel positioned between an obstruction in the vessel and tissue of the heart to be supplied with blood by the vessel. The step of forming includes placing a conduit in a heart wall between the chamber and the vessel with the first end of the conduit protruding into the chamber and protruding beyond an interior surface of the heart wall. The conduit includes a reinforcing wrap to resist crushing by contraction of the chamber and to help hold the conduit open.

### BRIEF DESCRIPTION OF THE DRAWINGS

FIG. 1 is a side sectional view of one embodiment of an implant shown in place in a human heart wall with the implant establishing a direct blood flow path from a heart chamber to a coronary vessel, constructed according to principles of this disclosure;

FIG. 2 is a top plan view of the implant depicted in FIG. 1;

FIG. 3 is a right end elevational view of the implant depicted in FIG. 2;

FIG. 4 is a rear elevational view of the implant depicted in FIG. 2;

FIG. 5 is a schematic, cross-sectional view of a portion of the wall of the implant in FIG. 2, the cross-section being taken along the line 5-5 of FIG. 2;

FIG. 6 is a schematic, top plan view of one embodiment of the implant, but shown before being bent;

FIG. 7 is another embodiment of the implant, the view being analogous to that shown in FIG. 6; and

FIG. 8 is another embodiment of the implant, the view being analogous to that shown in FIGS. 6 and 7.

### DETAILED DESCRIPTION OF THE PREFERRED EMBODIMENT

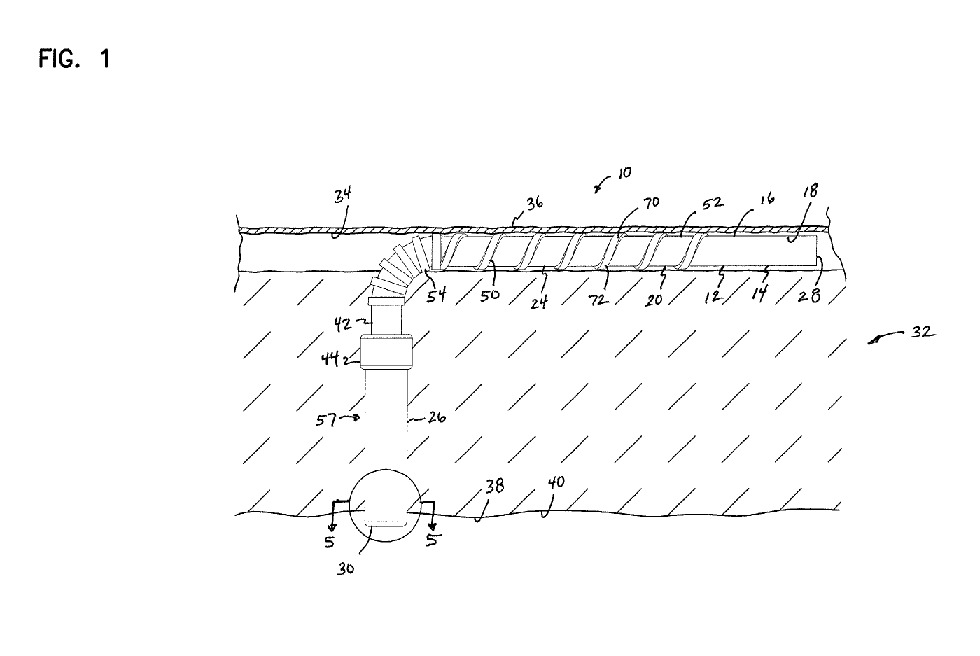

With initial reference to FIGS. 1-4, an implant is shown generally at 10. The implant 10 includes a composite of a hollow, rigid conduit 12. The conduit 12 includes a wall 14 defining an outer surface 16 and a hollow interior 18. In preferred embodiments, the wall 14 has a circular cross-section, forming a tube or cylinder 20. The conduit 12 includes a first portion 24, preferably corresponding to a vessel or vasculature portion, and a second portion 26, generally corresponding to a myocardial portion. The conduit 12 includes an open first end 28 that is defined by the vascular portion 24. The conduit 12 also includes an open second end 30 that is defined by the myocardial portion 26.

In FIG. 1, a cross-section of the myocardium 32 of a human heart is shown. As can be seen in FIG. 1, in preferred embodiments, the first portion 24 is dimensioned to be received within a lumen 34 of a coronary vasculature 36. As used herein, the term “vasculature” refers to veins or arteries. Note that the vasculature 36 resides exterior of the myocardium 32. The second portion 26 is dimensioned to extend from the vasculature 36 through the myocardium 32 and into a heart chamber 38. In preferred implementations, the heart chamber 38 will be the left ventricle 40. As can be seen in FIG. 1, the conduit 12 defines a blood flow pathway 42 within the interior 18 between the open first end 28 and the open second end 30. This allows for the flow of oxygenated blood directly from the left ventricle 40 through the pathway 42 and into the vasculature 36.

As discussed in the above-mentioned U.S. Pat. No. 5,984,956, the conduit 12 may be provided with tissue-growth inducing material 44 adjacent to an end 46 of the myocardial portion 26 to immobilize the myocardial portion 26 within the myocardium 32. The material 44 preferably surrounds the outer surface 16 of the wall 14, and may be a polyester woven sleeve or sintered metal to define pores into which tissue growth from the myocardium 32 may occur.

In preferred embodiments, the conduit 12 also includes a reinforcing wrap 50. The reinforcing wrap 50 extends along at least a section 52 of the outer surface 16 of the wall 14. In many preferred embodiments, the wrap 50 extends along at least a portion of the outer surface 16 of the wall 14 of the myocardial portion 26. The reinforcing wrap 50 helps to resist crushing of the conduit 12 in the myocardial portion 26, due to contraction of the chamber 38. Further, the reinforcing wrap 50 helps to hold open the blood flow pathway 42, in particular in the myocardial portion 26, during a part of the pump cycle when a vacuum effect or negative pressure runs through the interior 18 of the conduit 12. In many preferred embodiments, the reinforcing wrap 50 also extends along at least a portion of the outer surface 16 of the wall 14 of the vasculature portion 24. More details on certain preferred embodiments are described further below.

In preferred embodiments, the conduit 12 is formed of expanded polytetraflorethylene (ePTFE) material. A variety of materials may be used for the reinforcing wrap 50 including, generally, metallic or polymer. Of those materials possible, examples include nitinol, titanium, stainless steel and metal alloys. Other examples include a polymeric material, such as low density polyethylene.

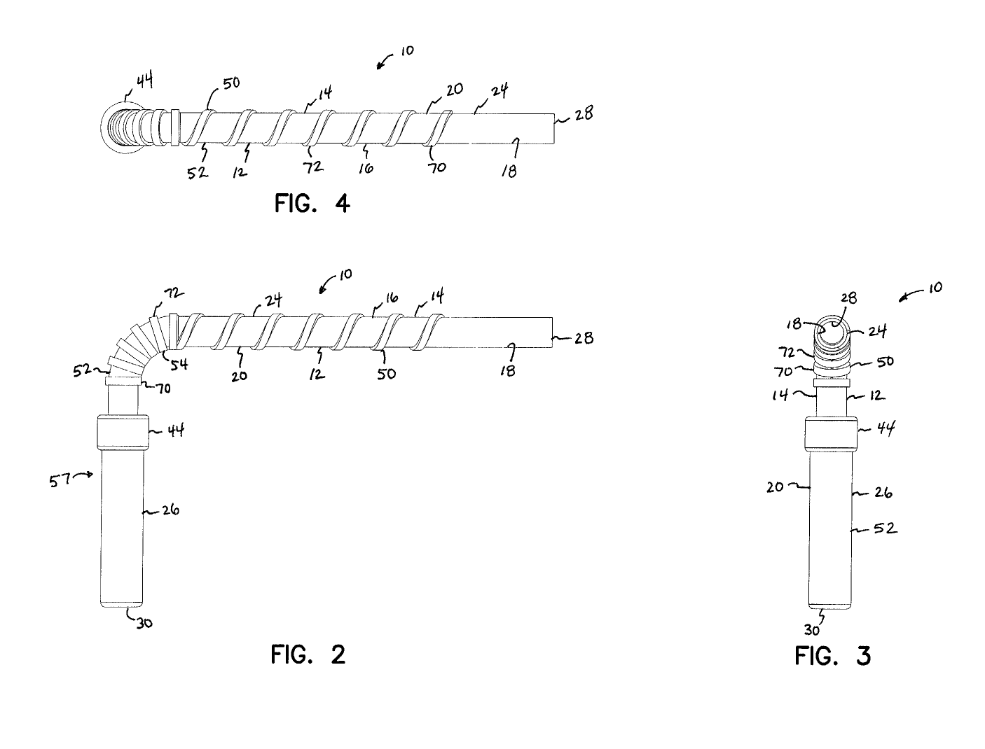

An interior radius 54 (FIGS. 1 and 2) is provided between the vasculature portion 24 and the myocardial portion 26, in preferred embodiments. The radius 54 provides support for the vasculature portion 24. In preferred embodiments, the vasculature portion 24 is angled relative to the myocardial portion 26 at an angle of between 30-150 degrees, preferably 90 degrees. It should be understood that an angle of a differing degree or no angle at all (that is, a straight conduit 12) could be used.

As mentioned above, the conduit 12 includes first end 28 and second end 30. The first end 28 extends in the lumen 34 of the vasculature 36. The second end 30 is inserted through the myocardium 32 and preferably projects and extends beyond the myocardium 32 to rest within the heart chamber 38.

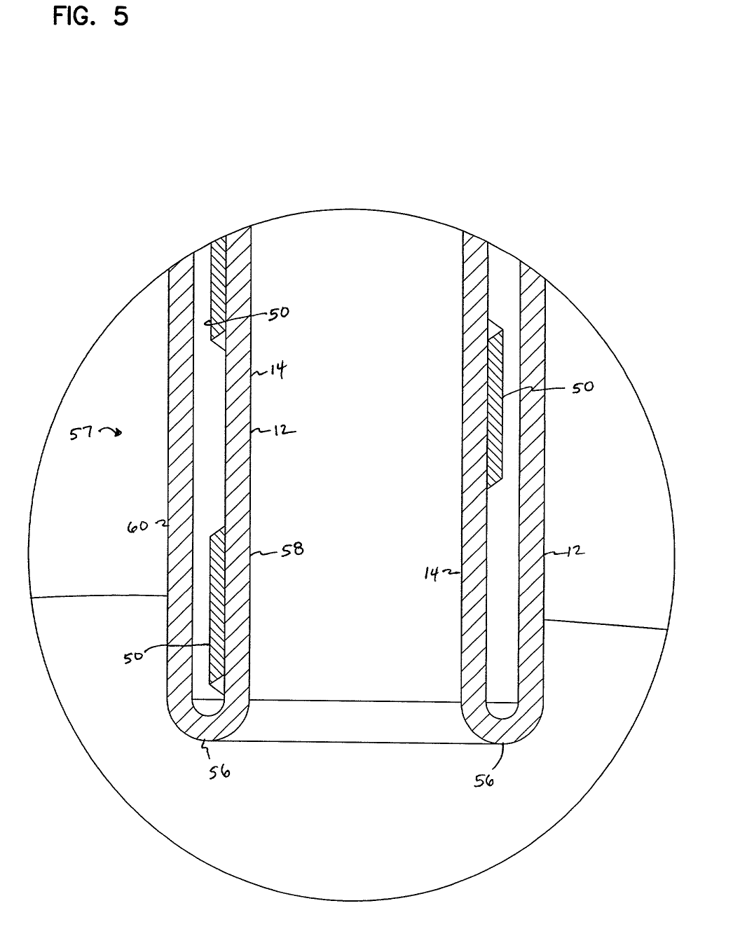

Attention is directed to FIG. 5. FIG. 5 is a cross-section of one part of the myocardial portion 26. In the particular preferred embodiment shown in FIG. 5, it can be seen that the wall 14 of the conduit 12 is wrapped back against itself in the myocardial portion 26 between the second end 30 and, in the embodiment shown, the cuff 44 to form an overlap region 57. The fold can be seen at 56. The wall 14 covers the reinforcing wrap 50 in overlap region 57, such that the reinforcing wrap 50 is sandwiched between an inner layer 58 and an outer layer 60 of the wall 14 along the myocardial portion 26. This particular construction helps to further strengthen the myocardial portion 26. Further, this construction helps to secure the wrap 50 to the wall 14.

In the embodiment shown in FIGS. 1-4, the reinforcing wrap 50 includes a band 70 that extends around the outer surface 16 of the wall 14. In preferred embodiments, the band 70 extends along each of the vasculature portion 24 and the myocardial portion 26. In the particular embodiment illustrated in FIGS. 1-4, the band 70 includes a coil or a spiral winding 72. The spiral winding 72 is formed by winding or coiling the band 70 around the outer surface 16 of the wall 14. Although no particular theory with respect to operation is asserted herein, it is believed that the spiral winding 72 increases the longitudinal strength of the conduit 12. Longitudinal strength is generally the strength that is parallel to the longitudinal axis of the conduit 12 (and is at right angles to the hoop strength, the hoop strength being tangent to the surface of the conduit 12).

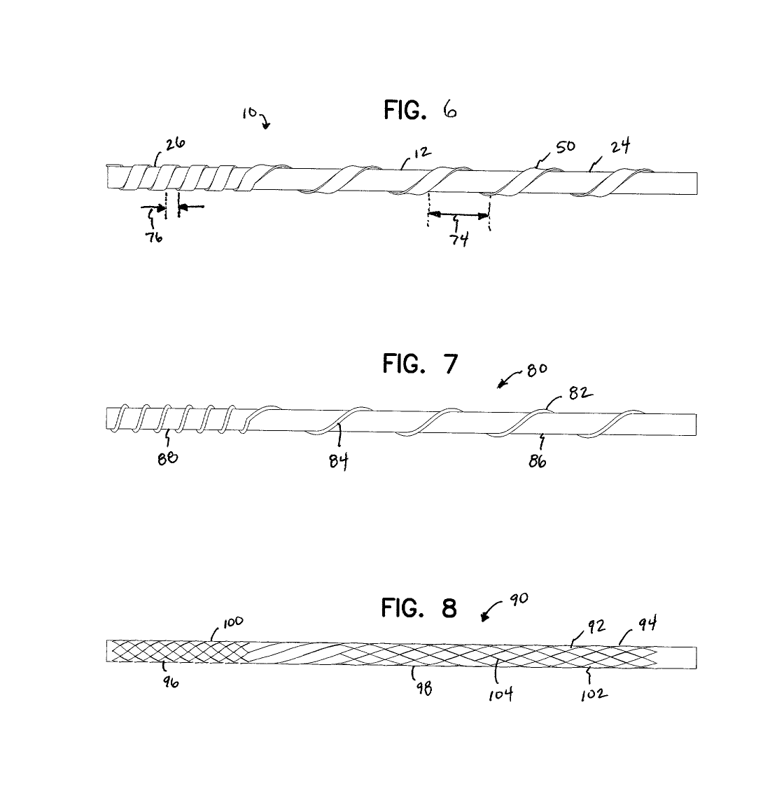

FIG. 6 shows the implant 10 of FIGS. 1-4, but before the conduit 12 has been bent into an angle and without having the fold 56 forming the overlap region 57. In FIG. 6, the band 70 is generally flat, defining a generally rectangular cross-section.

In preferred embodiments, the vasculature portion 24 is more flexible than the myocardial portion 26. In other words, the myocardial portion 26 is more rigid than the first portion 24. One way of accomplishing this preferred distinction in flexibility and rigidness is by having the spiral winding to wind with a pitch that is greater in the vasculature portion 24 than the myocardial portion 26. By the term “pitch”, it is meant the distance between two corresponding points on adjacent threads of the spiral winding 72. In particular, note that the spiral winding 72 around the vasculature portion 24 has a first pitch 74. The myocardial portion 26 has a second pitch 76. As can be seen in FIG. 6, the first pitch 74 is greater than the second pitch 76. Because the second pitch 76 is smaller than the first pitch 74, there is more reinforcing wrap 50 along the myocardial portion 26 than along the vasculature portion 24. This results in the myocardial portion 26 being stronger, more rigid, and less flexible than the vasculature portion 24.

FIG. 7 illustrates an alternative embodiment of the implant 10. The view in FIG. 7 is analogous to the view in FIG. 6. That is, an implant 80 shown in FIG. 7 is shown unbent and without an overlap region 57. The implant 80 in FIG. 7 also shows a spiral winding 82 of a band 84. In this embodiment, the band 84 is beaded or round. That is, the band 84 has a cross-section that is generally circular. Also note that in the FIG. 7 embodiment, the spiral winding 82 has different pitches in the vasculature portion 86 than in the myocardial portion 88. Again, in this embodiment, the pitch of the spiral winding 82 is greater in the vasculature portion 86 than in the myocardial portion 88.

In FIG. 8, another embodiment of an implant is shown generally at 90. The FIG. 8 embodiment is analogous to the embodiments of FIGS. 6 and 7. That is, in FIG. 8, the implant 90 includes a conduit 92 that is straight and unbent, but in preferred embodiments can be bent into an angle of about 90 degrees. The conduit 92 includes vasculature portion 94 and myocardial portion 96. In the FIG. 8 embodiment, the reinforcing wrap 98 includes a matrix 100 around at least a section of the wall 102 of the conduit 92. In the illustrated embodiment, the matrix 100 includes a netting or braid 104 covering at least a section of the myocardial portion 96. As can be seen in FIG. 8, in this embodiment, the braid 104 covers each of the vasculature portion 94 and the myocardial portion 96. In preferred embodiments, the braid 104 has a tighter weave in the myocardial portion 96 than in the vasculature portion 94. Again, this is done to result in greater rigidity in the myocardial portion 96 than in the vasculature portion 94.

As mentioned above, the reinforcing wrap 50 can be made from either a polymeric material or a metal. In certain preferred embodiments, the reinforcing wrap 50 (as well as the wrap 81 and wrap 98) comprises a plastically deformable material to allow selective bending of the vasculature portion 24 (or 86 or 94, as appropriate) into a desired shape. This is useful because when the surgeon is placing the implant 10, 80, 90 into place, after the myocardial portion 26, 88, 96 is in place, the vasculature portion 26, 86, 94 can be manipulated and moved to be bent or placed into the lumen 34 of the vasculature 36. Thus, in certain preferred embodiments, the reinforcing wrap 50 helps to hold the desired shape of the conduit 12. One useful material to accomplish this is nitinol.

To construct the implant 10, the reinforcing wrap 50 may be either thermally bonded, mechanically bonded, or a combination of each. For thermal bonding, any suitable thermal bonding agent may be used, such as silicone glue. The conduit 12 may be dipped in the silicone glue, then the wrap 50 may be applied. For example, the band 70 may be wound or coiled around the conduit wall 14. Alternatively, the band 70 may be dipped into the silicone glue and then wrapped around the conduit wall 14. A urethane bonding agent may also be utilized. Mechanical bonding, such as using the overlap 57 to shrink the conduit 12 and sandwich the wrap 50 can be used. Alternatively, cuffs can be used to secure the flap 50 to the conduit 12.

The spiral winding 72, 82 can be made by placing the conduit 12 on a turning lathe and turning the conduit relative to the band 70, 84, in order to wind the band 70, 84 onto the conduit 12. Alternatively, the spiral winding 72, 82 may be pre-made on a mandrel. After the winding is made, the conduit 12 may be inserted into the interior of the winding 72, 82.

To make the braid 104, in one embodiment, a plurality of wires, such as eight wires can be used and weaved into a braid. The braiding may be done around the conduit wall 14. Alternatively, the braid 104 can be pre-made, with the conduit 12 then inserted into the braid 104.

It should be understood that the embodiments of FIGS. 6-8 can also include an overlap region analogous to the overlap region 57 described and shown in FIGS. 1-4. Alternatively, it should be understood that the embodiment shown in FIGS. 1-4 may not include an overlap region 57, but may appear as shown in FIGS. 6-8.

In use, a method for performing a coronary vessel bypass procedure for supplementing a flow of blood to a coronary vessel can be performed using the implants described herein. The method would include forming a blood flow path from a heart chamber directly to the coronary vessel at a site in the vessel positioned between an obstruction in the vessel and tissue of the heart to be supplied with blood by the vessel. The step of forming includes placing a conduit, such as conduit 12, into a heart wall, between the chamber 38 and the vessel 36 with an end 30 of the conduit 12 protruding into the chamber 38 and beyond an interior surface 37 of the myocardium 32. Preferably, the conduit 12 includes reinforcing wrap 50 to resist crushing by contraction of the chamber 38 and to help hold open the conduit 12.

Preferably, the step of forming includes inserting the end 30 into the chamber 38 through the myocardium 32 and retaining the conduit 12 in the myocardium 32 and with the end 30 in blood flow communication with the blood within the chamber 38. Further, the end 28 of the conduit 12 is then connected to the coronary vessel or vasculature 36 for blood to flow from the end 28 substantially aligned with an axis of the lumen 34 of the vasculature 36.

In some preferred implementations, before the step of connecting the end 28 to the vasculature 36, the conduit 12 is plastically deformed into a desired shape to help connect the end 28 to the vasculature 36. In many preferred embodiments, the reinforcing wrap 50 will include a plastically deformable material to permit plastically deforming the conduit 12. As mentioned above, one useful plastically deformable material is nitinol.

It will be appreciated that modifications and equivalents may occur to one of ordinary skill in the art having the benefits of the teachings of principles of this disclosure. Many embodiments may be made according to the principles discussed herein.

What is claimed is:

1. A transmyocardial implant for establishing a blood flow path through a myocardium between a heart chamber and a lumen of a coronary vasculature residing at an exterior of the myocardium, the implant comprising:
(a) a hollow rigid conduit having: a wall defining an outer surface and an interior; an open first end; an open second end; a first portion defining said first end; a second portion defining said second end; and a reinforcing wrap;
(i) said first portion dimensioned to be received within the lumen and said second portion dimensioned to extend from the vasculature through the myocardium into the chamber;
(ii) said conduit defining a blood flow pathway within said interior between said first and second ends;
(iii) said second portion formed of a material sufficiently rigid to resist deformation and closure of said pathway in response to contraction of the myocardium; and
(iv) said reinforcing wrap extending along at least a section of the outer surface of the wall.

2. An implant according to claim 1 wherein:
(a) said reinforcing wrap extends along at least a portion of the outer surface of the wall of said second portion.

3. An implant according to claim 2 wherein:
(a) said reinforcing wrap extends along at least a portion of the outer surface of the wall of said second portion and said first portion.

4. An implant according to claim 1 wherein:
(a) said reinforcing wrap includes a band comprising a spiral winding around at least a section of said wall of said conduit.

5. An implant according to claim 4 wherein:
(a) said band comprises metal.

6. An implant according to claim 4 wherein:
(a) said band comprises a polymeric material.

7. An implant according to claim 4 wherein:
(a) said band comprises a spiral winding around at least a section of said second portion.

8. An implant according to claim 7 wherein:
(a) said band comprises a spiral winding around at least a section of said first portion and a section of said second portion;
(i) said spiral winding around said first portion defining a first pitch;
(ii) said spiral winding around said second portion defining a second pitch.

9. An implant according to claim 8 wherein:
(a) said first pitch is greater than said second pitch.

10. An implant according to claim 1 wherein:
(a) said reinforcing wrap includes a matrix around at least a section of said wall of said conduit.

11. An implant according to claim 10 wherein:
(a) said matrix comprises a braid covering at least a section of said second portion.

12. An implant according to claim 11 wherein:
(a) said band comprises a braid covering at least a section of said first portion and said second portion.

13. An implant according to claim 12 wherein:
(a) said braid has a tighter weave around said second portion than around said first portion.

14. An implant according to claim 1 wherein:
(a) said reinforcing wrap is sandwiched between layers of said wall along said second portion.

15. An implant according to claim 1 wherein:
(a) said reinforcing wrap extends along a section of each of said first portion and said second portion;
(i) said reinforcing wrap comprising a plastically deformable material to allow selective bending of said first portion into a desired shape.

16. An implant according to claim 15 wherein:
(a) said reinforcing wrap comprises a spiral band of nitinol.

17. An implant according to claim 1 wherein:
(a) said first portion is angled relative to said second portion at an angle of between 30-150 degrees.

18. An implant according to claim 1 wherein:
(a) said reinforcing wrap extends along the outer surface of the wall from said first end to said second end.

19. An implant according to claim 1 wherein:
(a) said reinforcing wrap is made from one of the group comprising nitinol, titanium, stainless steel, and metal alloys.

20. An implant according to claim 1 wherein:
(a) said reinforcing wrap is made from a polymeric material.

21. A method for making a transmyocardial implant for establishing a blood flow path through a myocardium between a heart chamber and a lumen of a coronary vasculature residing at an exterior of the myocardium; the method comprising:
(a) providing a hollow rigid conduit having: a wall defining an outer surface and an interior; an open first end; an open second end; a first portion defining the first end; and a second portion defining the second end;
(i) the first portion dimensioned to be received within the lumen and the second portion dimensioned to extend from the vasculature through the myocardium into the chamber; and
(b) providing a reinforcing wrap to extend along at least a section of the outer surface of the wall.

22. A method according to claim 21 wherein:
(a) said step of providing a reinforcing wrap includes spirally winding a band around the conduit.

23. A method according to claim 21 wherein:
(a) said step of providing a reinforcing wrap includes inserting the conduit into a pre-made spiral winding.

24. A method according to claim 21 wherein:
(a) said step of providing a reinforcing wrap includes providing a matrix around the conduit.

25. A method according to claim 24 wherein:
(a) said step of providing a matrix around the conduit includes inserting the conduit into a pre-made tubular woven braid.

26. A method according to claim 21 further including:
(a) thermally binding the reinforcing wrap to the conduit.

27. A method according to claim 21 further including:
(a) wrapping the conduit back to cover the reinforcing wrap and to sandwich the reinforcing wrap between layers of the conduit.

28. A method according to claim 21 further including:
(a) securing the reinforcing wrap to the conduit with at least one cuff.

29. A method for performing a coronary vessel bypass procedure for supplementing a flow of blood to a coronary vessel; the method comprising:
(a) forming a blood flow path from a heart chamber directly to the coronary vessel at a site in the vessel positioned between an obstruction in the vessel and tissue of the heart to be supplied with blood by the vessel;
(i) the forming including placing a conduit in a heart wall between the chamber and the vessel with a first end of the conduit protruding into the chamber and protruding beyond an interior surface of the heart wall;
(A) the conduit including a reinforcing wrap to resist crushing by contraction of the chamber and to help to hold the conduit open.

30. A method according to claim 29 wherein:
(a) said step of forming includes inserting the first end into the chamber through the heart wall and retaining the conduit in the heart wall and with the first end in blood flow communication with the blood within the chamber; and
(b) connecting the second end to the coronary vessel for blood to flow from the second end substantially aligned with an axis of a lumen of the coronary vessel.

31. A method according to claim 30 further including:
(a) before said step of connecting, plastically deforming the conduit into a desired shape to connect the second end to the coronary vessel;
(i) the reinforcing wrap comprising a plastically deformable material to permit plastically deforming of the conduit.
